(19) United States
(12) Patent Application Publication
(10) Pub. No.: US 2026/0345569 A1
(43) Pub. Date: Nov. 5, 2026

(54) OMEPRAZOLE-VALACYCLOVIR FORMULATIONS

(71) Applicant: Alec René Oliva, Redding, CA (US)

(72) Inventor: Alec René Oliva, Redding, CA (US)

(21) Appl. No.: 19/654,130

(22) Filed: Apr. 21, 2026

Related U.S. Application Data
(60) Provisional application No. 63/793,298, filed on Apr. 23, 2025.

(51) Int. Cl. A61K 31/4439; A61K 9/20; A61K 9/28; A61K 31/522

(52) U.S. Cl. CPC A61K 31/4439; A61K 9/2013; A61K 9/2027; A61K 9/2054; A61K 9/2846; A61K 31/522

(57) ABSTRACT

Pharmaceutical formulations comprising a proton-pump inhibitor and a nucleoside analogue are disclosed. In various embodiments, an enteric-coated tablet comprises omeprazole and valacyclovir for treating herpes viral infections, including herpes simplex virus type 1, herpes simplex virus type 2, and varicella-zoster virus infections. The enteric coating, comprising a copolymer of methacrylic acid and ethyl acrylate, resists dissolution in gastric juices and dissolves at intestinal pH, enabling systemic absorption of both active pharmaceutical ingredients through the intestinal mucosa. This intestinal absorption facilitates a synergistic antiviral effect between the proton-pump inhibitor and the nucleoside analogue.

### CROSS REFERENCE TO RELATED APPLICATIONS

This application claims priority to and the benefit of, and is a nonprovisional of, U.S. Patent Application No. 63/793,298, filed Apr. 23, 2025 and entitled “Omeprazole-Valacyclovir Formulations” the contents of which are incorporated by reference in their entirety for all purposes.

### FIELD

The disclosure relates to pharmaceutical formulations comprising proton-pump inhibitors and nucleoside analogues, and methods of use thereof. Various pharmaceutical formulations, including omeprazole-valacyclovir formulations, may be used to treat mammalian (e.g., human) viral infections, such as an infection with herpes viruses (e.g., herpes simplex virus type 1, herpes simplex virus type 2, and varicella-zoster virus). The formulations described herein may provide synergistic antiviral effects through co-administration of these active pharmaceutical ingredients.

### BACKGROUND

Human herpes viruses cause a variety of disease states in humans, including herpes labialis (infection of herpes simplex virus type 1 “HSV-1”), herpes zoster (infection from reactivation of varicella-zoster virus “VZV”), and genital herpes (infection of herpes simplex virus type 1 “HSV-2”). Valacyclovir is an antiviral therapy for, among other things, treating human herpes viral infections. Valacyclovir is a prodrug, being converted into the active form, acyclovir, by the liver. Acyclovir is then converted into acyclovir triphosphate, which inhibits viral replication. Acyclovir triphosphate is known to inhibit viral replication through at least one of three mechanisms: competitive inhibition of viral DNA polymerase, incorporation and termination of the growing viral DNA chain, and inactivation of the viral DNA polymerase.

Omeprazole is a proton-pump inhibitor used to treat peptic ulcer disease and gastrointestinal reflux disease, among other diseases of the digestive tract, in humans.

### SUMMARY

Various pharmaceutical formulations are disclosed herein. In various embodiments, a tablet comprising omeprazole and valacyclovir is provided. The tablet may further comprise an enteric coating. In various embodiments, a method for treating a herpes viral infection in a human includes administering a tablet comprising omeprazole and valacyclovir. In various embodiments, a tablet comprising a proton-pump inhibitor and a nucleoside analogue is provided. In various embodiments, the proton-pump inhibitor comprises at least one of omeprazole, esomeprazole, pantoprazole, lansoprazole, rabeprazole, and dexlansoprazole. In various embodiments, the nucleoside analogue is selected from the group consisting of valacyclovir, acyclovir, famciclovir, and penciclovir. In various embodiments, the tablet is configured for systemic absorption of the proton-pump inhibitor and the nucleoside analogue through intestinal mucosa. In various embodiments, the combination of the proton-pump inhibitor and the nucleoside analogue provides a synergistic antiviral effect.

### BRIEF DESCRIPTION OF THE DRAWINGS

The accompanying drawings are included to provide a further understanding of the disclosure and are incorporated in and constitute a part of this specification, illustrate embodiments of the disclosure, and together with the description serve to explain the principles of the disclosure.

FIG. 1 illustrates a method of manufacture, in accordance with various embodiments.

### DETAILED DESCRIPTION

According to various embodiments, a pharmaceutical formulation comprising omeprazole and valacyclovir. As described in detail here, various carriers, lubricants, and other excipients may be included. In various embodiments, omeprazole and valacyclovir formulations provide improved bioavailability, efficacy, and other improvements.

Specifically, various embodiments include coadministration of omeprazole and valacyclovir, via oral ingestion, leading to possible synergistic effects resulting in potentiation of antiviral activity, subsequently reducing severity and duration of ongoing herpetic outbreaks. Unlike the typical use of proton-inhibitors (PPIs), the mechanism of omeprazole's modulatory effect on acyclovir's action occurs on the cellular level rather than in the gastric mucosa. Therefore, various embodiments specifically utilize enteric coating technology to bypass absorption in the stomach, and deliver all or a majority of the dose of omeprazole to the intestinal mucosa, where systemic bioavailability will be higher than if administered for its antiacid effect, which entail absorption in the stomach to effect stomach acid secretion.

While effective, current monotherapies comprising of nucleoside analogues such as acyclovir and penciclovir, as well as their respective prodrugs valacyclovir and famciclovir, their activity can occasionally be limited by viral resistance patterns, poor uptake or overall reduced efficacy in some patients. Traditionally, patients endure 5-10 days of active infection, causing exceedingly painful and often unsightly lesions on the mouth or genitals for HSV, and torso for VZV.

However, animal and in vitro studies suggest that PPI's, particularly omeprazole, may significantly potentiate the efficacy of nucleoside analogues against HSV. Michaelis et al. (2019) demonstrated that omeprazole significantly enhances acyclovir's antiviral activity in HSV-1 and HSV-2 cell culture models by up to 10-fold, indicating omeprazole may act as a viral polymerase potentiator independent of acid suppression effects (Frontiers in Microbiology, 10:2790). No human trial has yet translated this synergy to clinical formulations. In various embodiments, an HSV treatment protocol is provided comprising concurrent administration of an antiviral medication and PPI in a short-course regimen. In various embodiments, an HSV treatment protocol is provided comprising concurrent administration of a nucleoside analog and PPI in a short-course regimen. In various embodiments, the nucleoside analog comprises at least one of valacyclovir, acyclovir, and/or famciclovir. In various embodiments, the PPI comprises at least one of omeprazole, esomeprazole, and/or pantoprazole. In various embodiments, an HSV treatment protocol is provided comprising concurrent administration of valacyclovir and omeprazole in a short-course regimen (e.g., valacyclovir 1 g+omeprazole 40 mg BID for 5-7 days). Enhanced clinical outcomes of administration of valacyclovir and omeprazole include faster lesion healing, reduced symptom duration, lower recurrence rates, and potentially lower dosing needs.

In various embodiments, other proton pump inhibitors besides omeprazole may be utilized in lieu of or in combination with omeprazole to achieve similar or enhanced synergistic antiviral effects upon coadministration with nucleoside analogues. PPIs share a common mechanism of action as inhibitors of the hydrogen-potassium ATPase enzyme system, and it is believed that their potential to potentiate antiviral activity may extend across the PPI class. Accordingly, esomeprazole, the S-enantiomer of omeprazole, may be substituted for racemic omeprazole in the formulations described herein with the expectation of comparable or improved synergistic effects due to its higher bioavailability and more predictable pharmacokinetic profile.

Similarly, pantoprazole, lansoprazole, rabeprazole, and dexlansoprazole may each be employed as the proton pump inhibitor component in the pharmaceutical formulations disclosed herein. While the specific potentiation characteristics may vary among different PPIs due to differences in their chemical structures, pKa values, and binding kinetics to the H+/K+ ATPase enzyme, each is expected to exhibit some degree of synergistic enhancement of nucleoside analogue antiviral activity based on the shared class mechanism. Pantoprazole, for example, may offer advantages in certain patient populations due to its lower potential for drug-drug interactions arising from reduced cytochrome P450 enzyme inhibition.

In various embodiments, combination PPI therapy may be employed wherein omeprazole is coadministered with one or more additional proton pump inhibitors. Such combination PPI approaches may be beneficial in patients who demonstrate suboptimal response to single-PPI regimens, or in cases where maximizing or tending to maximize systemic PPI exposure is desired to enhance the potentiation of antiviral activity. For example, a formulation comprising omeprazole and esomeprazole in combination with valacyclovir may provide enhanced synergistic effects compared to either PPI alone, potentially through complementary pharmacokinetic profiles or additive effects on intracellular pathways that potentiate nucleoside analogue activity.

Selection of the appropriate PPI or combination of PPIs for a given patient may depend on various factors including patient tolerability, potential drug-drug interactions with concomitant medications, hepatic function, and individual pharmacogenomic considerations such as CYP2C19 metabolizer status. In patients who are CYP2C19 poor metabolizers, PPIs that are less dependent on CYP2C19-mediated metabolism, such as rabeprazole, may be preferred to achieve more consistent plasma concentrations. In various embodiments, the pharmaceutical formulations may be tailored to substitute or combine PPIs based on individual patient characteristics to optimize both the antiviral synergy and overall therapeutic outcomes.

In various embodiments, other nucleoside analogues besides valacyclovir may be utilized in lieu of or in combination with valacyclovir to achieve similar or enhanced synergistic antiviral effects upon coadministration with proton pump inhibitors. Nucleoside analogues share a common mechanism of action as competitive inhibitors of viral DNA polymerase and chain terminators, and it is believed that the synergistic potentiation observed with PPI coadministration may extend across the nucleoside analogue class. Accordingly, famciclovir, the prodrug of penciclovir, may be substituted for valacyclovir in the formulations described herein with the expectation of comparable synergistic effects, particularly in patients who have demonstrated intolerance to valacyclovir or in cases of valacyclovir-resistant viral strains.

Acyclovir (the active metabolite of valacyclovir) may also be directly administered as the nucleoside analogue component in the pharmaceutical formulations disclosed herein. While valacyclovir offers improved oral bioavailability compared to acyclovir due to its prodrug nature, direct administration of acyclovir in combination with a PPI may be preferred in certain clinical scenarios, such as intravenous administration for severe infections or in patients with impaired hepatic function who may have reduced capacity to convert valacyclovir to acyclovir. Similarly, penciclovir, the active form of famciclovir, may be employed in topical formulations combined with PPIs for localized treatment of herpetic lesions.

In various embodiments, combination nucleoside analogue therapy may be employed wherein valacyclovir is co-administered with one or more additional nucleoside analogues in combination with a PPI. Such combination antiviral approaches may be beneficial in patients with severe or recurrent infections, in immunocompromised patients, or in cases of documented viral resistance to a single nucleoside analogue. For example, a formulation comprising valacyclovir and famciclovir in combination with omeprazole may provide enhanced antiviral efficacy through complementary mechanisms of action or by targeting resistant viral subpopulations, although careful consideration of potential additive toxicities should be given in connection with combining multiple nucleoside analogues.

Selection of the appropriate nucleoside analogue or combination of nucleoside analogues for a given patient may depend on various factors including the specific herpes virus being treated, patient tolerability, renal function, potential drug-drug interactions with concomitant medications, and viral resistance profiles. In patients with impaired renal function, dose adjustments or selection of nucleoside analogues with lower renal clearance may be appropriate to minimize accumulation and potential toxicity. In various embodiments, the pharmaceutical formulations may be tailored to substitute or combine nucleoside analogues based on individual patient characteristics and the specific viral infection to optimize both the PPI-potentiated antiviral synergy and overall therapeutic outcomes.

In various embodiments, the pharmaceutical formulations may further comprise additional therapeutic agents to address specific patient needs or to provide enhanced therapeutic outcomes. For patients experiencing pain associated with herpetic lesions, the formulation may include an analgesic such as acetaminophen or a non-steroidal anti-inflammatory drug (NSAID) such as ibuprofen. For immunocompromised patients, the formulation may include immunomodulatory agents that support immune function during active infection. For patients with concurrent bacterial superinfection of herpetic lesions, the formulation may include an antibiotic agent. The inclusion of such additional therapeutic agents may be implemented in the same tablet formulation or may be provided as a separate dosage form for concurrent administration with the PPI-nucleoside analogue formulation.

### DEFINITIONS

Unless otherwise specified, the following definitions apply.

Omeprazole may be known as 6-methoxy-2-[(4-methoxy-3,5-dimethyl-2-pyridinyl)methylsulfinyl]-1H. A chemical structure of omeprazole is depicted below:

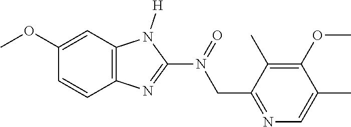

Valacyclovir may be known as 2-[(2-amino-6-oxo-1H-purin-9-yl)methoxy]ethyl (2S)-2-amino-3-methylbutanoate. A chemical structure of valacyclovir is depicted below:

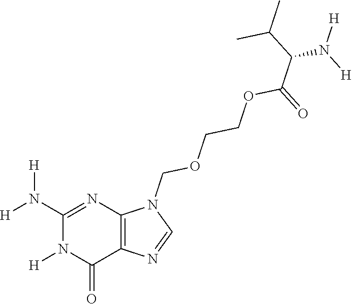 [SMILES: CC(C)[C@H](N)C(=O)OCCOCn1cnc2c(=O)[nH]c(N)nc21]

The term “administer,” “administration,” “deliver” or “delivery” (collectively “administration”), as used herein, means administration to the body via, without limitation, tablets, capsules, softgel capsules, injections, transdermal patches, creams, gels, or other mechanisms known in the art or hereinafter developed.

The term “therapeutically effective amount,” as used herein, means an amount of the pharmaceutical formulation sufficient to provide a therapeutic benefit to a patient, such as reduction in viral replication, acceleration of lesion healing, reduction in symptom severity, or reduction in symptom duration.

The term “proton-pump inhibitor” or “PPI,” as used herein, means a compound that inhibits the hydrogen-potassium adenosine triphosphatase (H+/K+ ATPase) enzyme system. Exemplary proton-pump inhibitors include, without limitation, omeprazole, esomeprazole, pantoprazole, lansoprazole, rabeprazole, and dexlansoprazole, and pharmaceutically acceptable salts, esters, and prodrugs thereof.

The term “nucleoside analogue,” as used herein, means a compound structurally similar to naturally occurring nucleosides that interferes with viral DNA synthesis. Exemplary nucleoside analogues include, without limitation, valacyclovir, acyclovir, famciclovir, penciclovir, ganciclovir, and valganciclovir, and pharmaceutically acceptable salts, esters, and prodrugs thereof.

The term “co-administer,” “coadministration,” “codeliver” or “codelivery” (collectively “coadministration”), as used herein, means administration to the body via, without limitation, tablets, capsules, softgel capsules, injections, transdermal patches, creams, gels, or other mechanisms known in the art or hereinafter developed, of at least two APIs at the same time (e.g., formulated in the same tablet) or within 5 minutes of one another to the body. The term “concurrent coadministration” means that at least two APIs are administered at the same time (e.g., formulated in the same tablet).

The term “gastric juices” means the watery, acidic digestive fluid that is secreted by various glands in the mucous membrane of the stomach and consists chiefly of hydrochloric acid, pepsin, rennin, and mucin.

The term, “API,” as used herein, refers to active pharmaceutical ingredient.

The term “excipients,” as used herein, refers to non-API substances such as carriers, solvents, lubricants and others used in formulating pharmaceutical products. They are generally safe for administering to humans according to established governmental standards, including those promulgated by the United States Food and Drug Administration.

The term “carrier,” as used herein, means any substance or mixture of substances that may be mixed with or contain an API.

The term “capsule,” as used herein, refers to a generally safe, readily dissolvable enclosure for carrying certain pharmaceutical products, and includes hard or soft shell capsules.

The term “softgel,” includes soft shell capsules, including soft-gelatin capsules and soft vegetable-based capsules, and soft capsules made from other materials providing the composition of such soft capsules are compatible with the formulations of the various embodiments described herein. A softgel may comprise two primary phases: a gel or vegetable-based capsule and a fill material of the pharmaceutical formulation as described herein.

The term “bioavailability,” as used herein means the concentration of an active ingredient (e.g., an API) in the blood (serum or plasma). The relative bioavailability may be measured as the concentration in the blood (serum or plasma) versus time. Other pharmacokinetic (PK) indicators may be used to measure and assess bioavailability, determined by suitable metrics including AUC, Cₘₐₓ and optionally Tₘₐₓ.

The terms “pharmacokinetics” and “pharmacokinetic measurements” include assessments and determinations to study absorption, distribution, metabolism, and excretion of a drug.

The term “AUC,” as used herein, refers to the area under the curve that represents changes in blood concentration of an API over time.

The term, “Cₘₐₓ” as used herein, refers to the maximum value of blood concentration shown on the curve that represents changes in blood concentrations of an API over time.

The term, “Tₘₐₓ” as used herein, refers to the time that it takes for API blood concentration to reach the maximum value.

Optionally, the term, “T_{1/2}” as used herein, refers to the time that it takes for API blood concentration to decline to one-half of the maximum level.

“Efficacy” means the ability of an API administered to a patient to produce a therapeutic effect in the patient.

“Tablet” refers to a pharmaceutical entity that is comprised of one or more APIs which is administered to, or taken by, a patient.

Collectively AUC, Cₘₐₓ, and optionally Tₘₐₓ and T_{1/2}, are the principle pharmacokinetic parameters that can characterize the pharmacokinetic responses of a particular drug product such as omeprazole or valacyclovir in an animal or human subject.

### DESCRIPTION

Generally, the pharmaceutical formulations described herein are prepared and administered as tablets, such as enteric coated tablets.

As described above, it has been discovered that combination therapies of valacyclovir and omeprazole lead to surprisingly increased efficacy of valacyclovir as an antiviral therapeutic agent. Conventionally, omeprazole is a proton pump inhibitor (PPI) intended to act upon parietal cells in the stomach lining that secrete gastric juices, including hydrochloric acid, by binding to and inhibiting the hydrogen-potassium adenosine triphosphatase (H+/K+ ATPase) enzyme system, thereby downregulating such secretions. PPIs such as omeprazole are commonly prescribed for treatment of gastroesophageal reflux disease (GERD), peptic ulcer disease, and Zollinger-Ellison syndrome. However, in vitro studies, referenced above, have found that coadministration of valacyclovir and omeprazole to cells directly demonstrate an increased efficacy of the antiviral properties of valacyclovir. Without being bound by theory, it is believed that omeprazole may enhance the bioavailability of valacyclovir or potentiate its antiviral activity through modulation of intracellular pathways unrelated to gastric acid suppression. This suggests that the omeprazole's synergistic effects are unrelated to omeprazole's activity relative to digestive tract tissues, and instead may involve direct cellular mechanisms that enhance valacyclovir's conversion to acyclovir or its incorporation into viral DNA polymerase inhibition pathways.

To that end, conventional administration of omeprazole, wherein the compound is absorbed primarily through gastric tissues and exerts its pharmacological effect locally on parietal cells, would be insufficient to elicit the desired synergistic effects between omeprazole and valacyclovir. Since the synergistic mechanism appears to entail systemic coadministration and absorption of both compounds, formulations that allow omeprazole to bypass gastric absorption and instead be absorbed in the intestinal tract are of particular interest. Thus, in accordance with various embodiments, an enteric coating is disposed on a tablet containing both valacyclovir and omeprazole. The enteric coating, as described above, is resistant to dissolution in the acidic environment of the gastric juices (typically pH 1.5 to 3.5) and may thus protect the omeprazole from premature release and absorption in the stomach. By preventing gastric release, the enteric coating ensures that the omeprazole does not exert its conventional proton pump inhibition activity on local gastric tissues, but rather remains encapsulated until the tablet reaches the higher pH environment of the intestines (typically pH 6 to 7.4). The enteric coating is then degraded in the intestines so that the valacyclovir and omeprazole may be absorbed systemically through the intestinal mucosa and enter systemic circulation together, thereby enabling the synergistic antiviral effect to be realized in target tissues throughout the body.

In various embodiments, the tablet core may be configured with a bilayer or multilayer architecture to optimize the release characteristics of each API. A bilayer tablet may comprise a first layer containing omeprazole and a second layer containing valacyclovir, which may allow for different release profiles for each API. A multilayer tablet may further comprise intermediate barrier layers or additional API-containing layers to achieve sequential or pulsatile release. In various embodiments, the tablet core may comprise a matrix formulation wherein the APIs are dispersed within a polymeric matrix that controls the rate of drug release. The matrix may comprise hydrophilic polymers such as hydroxypropyl methylcellulose (HPMC) or hydrophobic polymers such as ethylcellulose, or combinations thereof.

A tablet may be compressed or molded in its manufacture, and a tablet may be of almost any size, shape, weight, and color. Most tablets are intended to be swallowed whole and accordingly, tablets according to the disclosure are designed for oral administration. In various embodiments, the tablet according to the disclosure is a round tablet. Round tablets may have a diameter in the range of about 0.04 inches to about 1.18 inches (about 1 mm to about 30 mm), in particular in the range of about 0.08 inches to about 0.98 inches (about 2 mm to about 25 mm), more in particular about 0.20 inches to about 0.91 inches (about 5 mm to about 23 mm), even more in particular about 0.28 inches to about 0.51 inches (about 7 mm to about 13 mm); and a thickness in the range of about 0.04 inches to about 0.47 inches (about 1.0 mm to about 12 mm), in particular in the range of about 0.08 inches to about 0.39 inches (about 2.0 mm to about 10 mm), even more in particular from 0.12 inches to about 0.35 inches (3.0 mm to about 9.0 mm), even further in particular from about 0.16 inches to about 0.31 inches (about 4.0 mm to about 8.0 mm). In various embodiments, the tablet according to the disclosure is an oblong tablet. Oblong tablets preferably have a lengthwise extension (longitudinal extension) of about 0.04 inches to about 1.18 inches (about 1 mm to about 30 mm), in particular in the range of about 0.08 inches to about 0.98 inches (about 2 mm to about 25 mm), more in particular about 0.20 inches to about 0.91 inches (about 5 mm to about 23 mm), even more in particular about 0.28 inches to about 0.79 inches (about 7 mm to about 20 mm); a width in the range of about 0.04 inches to about 1.18 inches (about 1 mm to about 30 mm), in particular in the range of about 0.08 inches to about 0.98 inches (about 2 mm to about 25 mm), more in particular about 0.20 inches to about 0.91 inches (about 5 mm to about 23 mm), even more in particular about 0.28 inches to about 0.51 inches (about 7 mm to about 13 mm); and a thickness in the range of about 0.04 inches to about 0.47 inches (about 1.0 mm to about 12 mm), in particular in the range of about 0.08 inches to about 0.39 inches (about 2.0 mm to about 10 mm), even more in particular from 0.12 inches to about 0.35 inches (3.0 mm to about 9.0 mm), even further in particular from about 0.16 inches to about 0.31 inches (about 4.0 mm to about 8.0 mm).

The tablets according to the disclosure can optionally be provided, partially or completely, with a coating. The tablets according to the disclosure may comprise film coated with conventional film coating compositions. Suitable coating materials are commercially available, e.g., polymeric coatings (e.g., those sold under the trademarks Opadry® and Eudragit®). Examples of suitable coating materials include cellulose derivatives such as cellulose esters and cellulose ethers, for example methylcellulose (MC), hydroxypropylmethylcellulose (HPMC), hydroxypropylcellulose (HPC), hydroxyethylcellulose (HEC), and sodium carboxymethylcellulose (Na-CMC); acrylic polymers such as poly(meth)acrylates, for example aminoalkylmethacrylate copolymers, methacrylic acid methylmethacrylate copolymers; vinyl polymers such as polyvinylpyrrolidone, polyvinyl alcohol, and polyvinylacetate; and natural film formers such as shellac, zein, and hydroxypropyl starch. In various embodiments, the coating is water-soluble. In various embodiments, the coating is based on polyvinyl alcohol, such as polyvinyl alcohol-partially hydrolyzed, and may additionally contain polyethylene glycol, such as macrogol 3350, and/or pigments. In various embodiments, the coating is based on hydroxypropylmethyl cellulose, such as hypromellose type 2910. The coating can be resistant to gastric juices and dissolves in response to a change in the pH value of the release environment. By means of this coating, it is possible to ensure that the tablet according to the disclosure in various embodiments passes through the stomach undissolved and the active compound is only, or substantially, released in the intestines. The coating which is resistant to gastric juices dissolves at a pH value of between 5 and 7.5. In various embodiments, the enteric coating is comprised of one or more compositions comprising copolymers of methacrylic acid and ethyl acrylate, and copolymers of methacrylic acid and ethyl acrylate to which a monomer of methylacrylate has been added during polymerization.

In various embodiments, the coating described above may be an enteric coating specifically designed to protect the active pharmaceutical ingredients from the acidic environment of the stomach and to ensure targeted release in the intestinal tract. The enteric coating provides a pH-dependent barrier that remains intact at the low pH values characteristic of gastric fluid (typically pH 1.0 to 3.5) but dissolves or becomes permeable at the higher pH values found in the duodenum and small intestine (typically pH 5.5 to 7.4). This pH-responsive behavior is important for the omeprazole-valacyclovir formulations disclosed herein, as it enables systemic absorption of both APIs through the intestinal mucosa rather than gastric absorption, thereby facilitating the synergistic antiviral effect described above.

The polymeric coating family of polymers (e.g., those sold under the trademark Eudragit®), referenced above, includes several grades specifically formulated for enteric coating applications. A methacrylic acid-ethyl acrylate copolymer (1:1) (e.g., that sold under the trademark Eudragit® L100-55) dissolves at pH values above 5.5 and is suitable for targeting drug release to the duodenum. A methacrylic acid-methyl methacrylate copolymer (e.g., that sold under the trademark Eudragit® L100) dissolves at pH values above 6.0 and provides release in the jejunum. A methacrylic acid-methyl methacrylate copolymer with a higher methyl methacrylate content (e.g., that sold under the trademark Eudragit® S100) dissolves at pH values above 7.0 and may be used for targeted release in the ileum or colon. Selection among these enteric coating grades allows for precise control over the site of drug release within the gastrointestinal tract.

In addition to the Eudragit® polymers, other enteric coating materials suitable for use in the formulations disclosed herein include cellulose-based enteric polymers. Cellulose acetate phthalate (CAP) is a widely used enteric coating material that dissolves at pH values above approximately 6.0. Hydroxypropyl methylcellulose phthalate (HPMCP), available in grades HP-50 and HP-55, provides dissolution at pH values above 5.0 and 5.5, respectively. Hydroxypropyl methylcellulose acetate succinate (HPMCAS), available in L, M, and H grades, offers dissolution thresholds ranging from pH 5.5 to 6.8. These cellulose-based enteric polymers may be used alone or in combination with methacrylic acid copolymers to achieve desired release profiles.

In various embodiments, the enteric coating may comprise a combination of enteric polymers to achieve customized dissolution profiles or to improve coating performance characteristics. For example, a blend of a polymeric coating (e.g., that sold under the trademark Eudragit® L100-55) and a polymeric coating (e.g., that sold under the trademark Eudragit® S100) may be employed to provide a gradual release profile that begins in the duodenum and continues through the ileum. In other embodiments, enteric polymers may be combined with plasticizers such as triethyl citrate, dibutyl sebacate, or polyethylene glycol to improve film flexibility and reduce brittleness. The enteric coating may further comprise anti-tacking agents such as talc or glyceryl monostearate, and opacifiers or colorants such as titanium dioxide or iron oxides for product identification purposes. The total enteric coating weight gain may range from about 1% to about 20% of the tablet core weight, more particularly about 2% to about 15%, and even more particularly about 4% to about 10%, with coating thicknesses selected to ensure adequate gastric protection while permitting timely intestinal release.

In various embodiments, the enteric coating may comprise one or more other polymeric materials that achieve pH-dependent dissolution. Polyvinyl acetate phthalate (PVAP) dissolves at pH values above approximately 5.0 and may be used alone or in combination with the methacrylic acid copolymers described above. Shellac, a natural polymer, provides enteric protection with dissolution at pH values above approximately 7.0 and may be employed in formulations where natural-origin excipients are preferred. Zein, a prolamine protein derived from corn, exhibits pH-responsive behavior and may be used as an enteric coating material or in combination with synthetic enteric polymers. The selection of enteric coating material or combination of materials may be tailored based on the desired release site within the gastrointestinal tract, manufacturing considerations, and patient population specifications.

Exemplary dosage strengths for valacyclovir for use in the formulations described herein include, without limitation, about 250 mg to about 3000 mg, more particularly about 500 mg to about 2000 mg, and even more particularly about 750 mg to about 1500 mg, where the term “about” in this context only means +/−10 mg. Specific examples include 500 mg, 1000 mg, and 2000 mg. Dose administration may include once per day (q.d.), twice per day (b.i.d) three times per day (t.i.d.) and four times per day (q.i.d). In various embodiments, dose strength is 1000 mg administered b.i.d. for five to seven days.

Exemplary dosage strengths for omeprazole for use in the formulations described herein include, without limitation, about 10 mg to about 80 mg, more particularly about 20 mg to about 60 mg, and even more particularly about 30 mg to about 50 mg, where the term “about” in this context only means +/−2 mg. Specific examples include 20 mg, 40 mg, and 60 mg. Dose administration may include once per day (q.d.), twice per day (b.i.d) three times per day (t.i.d.) and four times per day (q.i.d). In various embodiments, dose strength is 40 mg administered b.i.d. for five to seven days. In various embodiments, dose strength is 60 mg administered b.i.d. for five to seven days.

Exemplary dosage strengths for omeprazole and valacyclovir for use in the formulations described herein include, without limitation, are described in TABLE 1.

TABLE 1

| Omeprazole (mass) | Valacyclovir (mass) | Administration | Duration |
|---|---|---|---|
| 20 | 500 | bid | 5 days |
| 20 | 500 | bid | 6 days |
| 20 | 500 | bid | 7 days |
| 40 | 1000 | bid | 5 days |
| 40 | 1000 | bid | 6 days |
| 40 | 1000 | bid | 7 days |
| 60 | 2000 | bid | 5 days |
| 60 | 2000 | bid | 6 days |
| 60 | 2000 | bid | 7 days |

Particular dosages and administration are dependent upon, among other factors, patient weight, renal function, and infection severity.

In various embodiments, the dosage and administration regimen may be adjusted based on the specific herpes virus being treated. For herpes labialis caused by HSV-1, a shorter treatment course of three to five days may be sufficient. For genital herpes caused by HSV-2, a treatment course of five to seven days may be appropriate. For herpes zoster caused by VZV reactivation, a longer treatment course of seven to ten days may be indicated. For immunocompromised patients or patients with severe infections, treatment may be extended to fourteen days or longer.

In various embodiments, combination therapies containing different PPIs and other nucleoside analogues include, but are not limited to, combinations of the below:

TABLE 2

| | Nucleoside | | |
|---|---|---|---|
| | Analogues | Adminis- | |
| PPI (mass) | (mass) | tration | Duration |
| Omeprazole (20 mg) | Famciclovir (500 mg) | bid | 5 days |
| Omeprazole (20 mg) | Famciclovir (500 mg) | bid | 6 days |
| Omeprazole (20 mg) | Famciclovir (500 mg) | bid | 7 days |
| Omeprazole (40 mg) | Famciclovir (500 mg) | bid | 5 days |
| Omeprazole (40 mg) | Famciclovir (500 mg) | bid | 6 days |
| Omeprazole (40 mg) | Famciclovir (500 mg) | bid | 7 days |
| Omeprazole (60 mg) | Famciclovir (500 mg) | bid | 5 days |
| Omeprazole (60 mg) | Famciclovir (500 mg) | bid | 6 days |
| Omeprazole (60 mg) | Famciclovir (500 mg) | bid | 7 days |
| Esomeprazole (40 mg) | Famciclovir (500 mg) | bid | 5 days |
| Esomeprazole (40 mg) | Famciclovir (500 mg) | bid | 6 days |
| Esomeprazole (40 mg) | Famciclovir (500 mg) | bid | 7 days |
| Pantoprazole (40 mg) | Acyclovir (800 mg) | bid | 5 days |
| Pantoprazole (40 mg) | Acyclovir (800 mg) | bid | 6 days |
| Pantoprazole (40 mg) | Acyclovir (800 mg) | bid | 7 days |
| Pantoprazole (40 mg) | Famciclovir (500 mg) | bid | 5 days |
| Pantoprazole (40 mg) | Famciclovir (500 mg) | bid | 6 days |
| Pantoprazole (40 mg) | Famciclovir (500 mg) | bid | 7 days |
| Esomeprazole (40 mg) | Acyclovir (800 mg) | bid | 5 days |
| Esomeprazole (40 mg) | Acyclovir (800 mg) | bid | 6 days |
| Esomeprazole (40 mg) | Acyclovir (800 mg) | bid | 7 days |

The combinations in TABLE 2 thus serve as various embodiments for patients with differing tolerability and/or therapeutic response profiles.

In various embodiments, the pharmaceutical formulations may be administered in combination with other therapeutic agents as part of a comprehensive treatment regimen. For patients experiencing significant pain associated with herpetic infections, particularly herpes zoster, the formulation may be co-administered with analgesic agents such as acetaminophen, non-steroidal anti-inflammatory drugs (NSAIDs), or neuropathic pain medications such as gabapentin or pregabalin. For patients with secondary bacterial infection of herpetic lesions, the formulation may be co-administered with appropriate antibiotic agents. For immunocompromised patients, the formulation may be co-administered with immunomodulatory agents.

In additional embodiments, a pharmaceutical formulation comprises omeprazole, valacyclovir and at least one carrier, and other pharmaceutically acceptable excipients.

In various embodiments, the pharmaceutical formulation may further comprise a stabilizer to protect the APIs from degradation during storage. Suitable stabilizers include antioxidants such as butylated hydroxytoluene (BHT), butylated hydroxyanisole (BHA), ascorbic acid, and alpha-tocopherol; chelating agents such as ethylenediaminetetraacetic acid (EDTA) and citric acid; and pH-buffering agents such as sodium phosphate, potassium phosphate, and sodium citrate. The stabilizer may be present in an amount of about 0.001% to about 2% w/w, more particularly about 0.010% to about 10% w/w, and even more particularly about 0.0500 to about 0.500 w/w, based on the total weight of the formulation.

In various embodiments, a tablet may comprise the constituents listed in TABLE 3 below. In various embodiments, the tablet may include a flavoring agent or sweetener for improved palatability in pediatric patients.

TABLE 3

| | | Relative or | |
|---|---|---|---|
| | | Absolute | |
| Material | Function | amount | |
| Omeprazole | Active | 40 mg (or any suitable | |
| | | dose set forth herein) | |
| Valacyclovir | Active | 1000 mg (or any suitable | |
| | | dose set forth herein) | |
| Microcrystalline | Filler | Up to 40% w/w | |
| cellulose | | | |
| Croscarmellose | Disintegrant | 2%-5% | w/w |
| sodium | | | |
| Magnesium stearate | Lubricant | 0.5%-1% | w/w |
| Povidone K30 | Binder | 3%-6% | w/w |
| (polyvinylpyrrolidone) | | | |
| Glycerin | Humectant | 0.1%-25% | w/w |
| Triethyl citrate | Plasticizer (for use | 0.01%-5% | w/w |
| | with coating) | | |
| Polymeric coating | Coating | 0.1%-5% | w/w |

such as methacrylic
acid-ethyl acrylate
copolymer (1:1) (sold
as EUDRAGIT L100-
55)

In various embodiments, a tablet may comprise the constituents listed in TABLE 4 below:

TABLE 4

| | | Relative or | |
|---|---|---|---|
| | | Absolute | |
| Material | Function | amount | |
| Formulation A (Low Dose) | | | |
| Omeprazole | Active | 20 | mg |
| Valacyclovir | Active | 500 | mg |
| Microcrystalline cellulose | Filler | Up to 40% w/w | |
| Croscarmellose sodium | Disintegrant | 2%-5% | w/w |
| Magnesium stearate | Lubricant | 0.5%-1% | w/w |
| Povidone K30 | Binder | 3%-6% | w/w |
| Glycerin | Humectant | 0.1%-25% | w/w |
| Triethyl citrate | Plasticizer | 0.01%-5% | w/w |
| Polymeric coating such as | Coating | 0.1%-5% | w/w |
| methacrylic acid-ethyl acrylate | | | |
| copolymer (e.g., that sold | | | |
| under the trademark EUDRAGIT | | | |
| L100-55) | | | |
| Formulation B (Standard Dose) | | | |
| Omeprazole | Active | 40 | mg |
| Valacyclovir | Active | 1000 | mg |
| (same excipients as Formulation A) | | | |
| Formulation C (High Dose) | | | |
| Omeprazole | Active | 60 | mg |
| Valacyclovir | Active | 2000 | mg |
| (same excipients as Formulation A) | | | |

The choice of excipient will, to a large extent, depend on factors such as the particular mode of administration, the effect of the excipient on solubility and stability, and the nature of the dosage form. Excipients used in various embodiments may include colorants, flavoring agents, preservatives and taste-masking agents. Colorants, for example, may comprise about 0.1% to about 2% by weight. Preservatives may comprise methyl and propyl paraben, for example, in a ratio of about 10:1, and at a proportion of about 0.005% and 0.05% by weight.

Colorants can be used for marketing and product identification/differentiation purposes. Suitable colorants include synthetic and natural dyes and combinations thereof.

Flavorings can be used to mask unpleasant odors and tastes of fill formulations. Suitable flavorings include synthetic and natural flavorings. The use of flavorings can be problematic due to the presence of aldehydes which can cross-link gelatin. As a result, buffering salts and acids can be used in conjunction with flavorings that contain aldehydes in order to minimize cross-linking of the gelatin.

Formulations in accordance with various embodiments may be administered orally. Oral administration may involve swallowing, so that the compound enters the gastrointestinal tract, or buccal or sublingual administration may be employed by which the compound enters the blood stream directly from the mouth.

In various embodiments, the pharmaceutical formulation may be provided in dosage forms other than tablets. Capsule formulations may comprise hard gelatin capsules or hard capsules made from hydroxypropyl methylcellulose (HPMC), wherein the capsule contains a powder or granule fill comprising the APIs and excipients. Softgel formulations may comprise the APIs dissolved or suspended in a liquid fill material, such as polyethylene glycol, medium-chain triglycerides, or vegetable oils, enclosed within a soft gelatin shell. Powder formulations for reconstitution may comprise the APIs and excipients in a dried form that is reconstituted with water or another suitable liquid prior to administration. Each of these additional dosage forms may further comprise an enteric coating or be formulated for enteric release to achieve the synergistic effects described herein.

In various embodiments, the pharmaceutical formulation may be provided as a kit comprising multiple dosage units for a complete treatment course. The kit may comprise sufficient tablets for administration twice daily for a treatment period of five to seven days, or for a treatment period of up to fourteen days for immunocompromised patients or patients with severe infections. The kit may further comprise patient instructions for use, including dosing schedules and information regarding expected treatment outcomes. In various embodiments, the kit may comprise tablets of different dosage strengths to allow for dose titration based on patient response or tolerability.

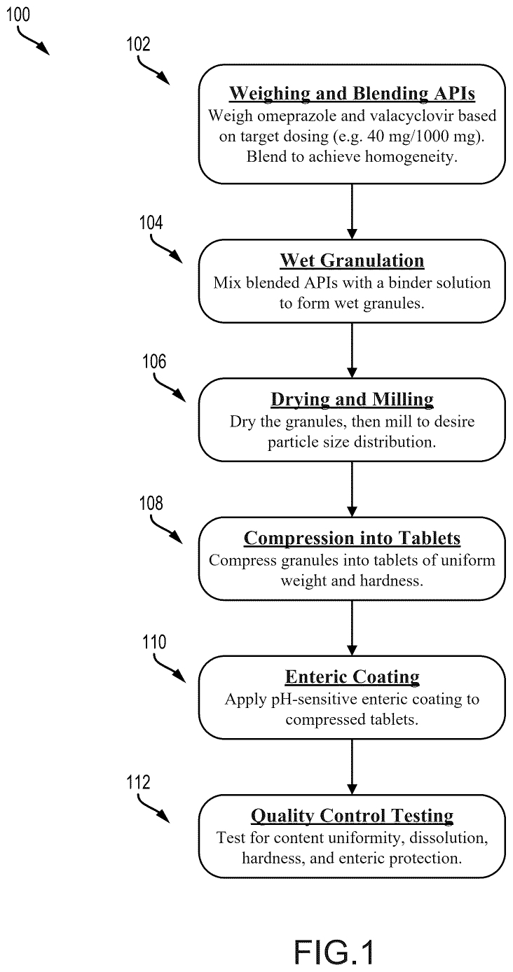

With reference to FIG. 1, a method of manufacture 100 is illustrated. In step 102, weighing and blending of APIs is performed. In step 102, the PPI and nucleoside analogue, typically in powdered form, are weighed individually and then blended together. For example, 40 mg of omeprazole and 1000 mg of valacyclovir are weighed and blended to achieve homogeneity. Blending may occur by agitation, stirring, or other suitable means.

In step 104, the mixed API is blended with a binder solution to form wet granules. The binder may be, for example, polyvinylpyrrolidone along with other suitable solvents or agents, in various embodiments. The wet granules are then dried and milled in step 106 to form a powder of desired particle size distribution. Typically, particle size distribution is fairly uniform and consistent with a powder form. In step 108, the ground granules are compressed into tablets of uniform height and hardness. Compression may be accomplished in any suitable manner, for example, pressing in a mold or using a tablet press.

The resultant tablets are enteric coated in step 110. As discussed above, methacrylic acid-ethyl acrylate copolymer may be used to coat the tablets. The enteric coating resists dissolution below certain pH levels, such as the pH levels found in the stomach. By resisting dissolution at low pH (e.g., less than pH 4), the APIs in the tablet are not absorbed systemically until entrance into the intestines.

Quality control testing 112 includes monitoring coated tablets for uniformity in size, hardness, enteric protection, and dissolution. Testing and monitoring may occur in any commonly acceptable commercial manner.

In various embodiments, the pharmaceutical formulations may be packaged in unit-dose packaging such as blister packs to protect the tablets from moisture and to facilitate patient compliance. The blister pack may comprise a calendar-style layout indicating the day and time of administration for each tablet. The packaging may further comprise a desiccant to maintain low humidity within the package. In various embodiments, the packaging may be child-resistant to prevent accidental ingestion by children.

In various embodiments, the pharmaceutical formulation may be prepared using other manufacturing processes. Direct compression methods may be employed wherein the APIs and excipients are blended and compressed into tablets without a prior granulation step, which may be suitable for formulations where the APIs exhibit acceptable flow and compressibility characteristics. Dry granulation methods, such as roller compaction, may be used to improve powder flow and compressibility while avoiding exposure of the APIs to water or heat. Fluid bed granulation may be employed in lieu of high-shear wet granulation, wherein the binder solution is sprayed onto the powder blend while the particles are suspended in a stream of air. Hot melt extrusion may be utilized for preparing solid dispersions of the APIs, which may enhance dissolution and bioavailability. The selection of manufacturing process may be based on the physicochemical properties of the APIs, desired release characteristics, and scale of production.

In various embodiments, the pharmaceutical formulation may be manufactured using continuous manufacturing processes. Continuous wet granulation, continuous drying, and continuous tablet compression may be employed to achieve consistent product quality and improved manufacturing efficiency. Process analytical technology (PAT) may be employed to monitor critical quality attributes in real-time during continuous manufacturing. In various embodiments, the manufacturing process may incorporate quality by design (QbD) principles, wherein critical process parameters are identified and controlled to ensure that the finished product meets predetermined quality specifications.

It will be apparent to those skilled in the art that various modifications and variations can be made in the present disclosure without departing from the spirit or scope of the disclosure. Thus, it is intended that the present disclosure cover the modifications and variations of this disclosure provided they come within the scope of the appended claims and their equivalents.

In various embodiments, the pharmaceutical formulations disclosed herein may be used prophylactically to prevent or reduce the frequency of recurrent herpetic outbreaks. For patients with frequent recurrences, a suppressive therapy regimen comprising daily administration of the pharmaceutical formulation may be employed. The suppressive therapy dosage may be lower than the dosage used for treatment of acute outbreaks. For example, a suppressive therapy regimen may comprise omeprazole at about 20 mg and valacyclovir at about 500 mg administered once daily. In various embodiments, suppressive therapy may be continued for periods of six months to one year or longer, with periodic evaluation of the need for continued suppression.

Likewise, numerous characteristics and advantages have been set forth in the preceding description, including various options together with details of the structure and function of the devices and/or methods. The disclosure is intended as illustrative only and as such is not intended to be exhaustive. It will be evident to those skilled in the art that various modifications may be made, especially in matters of structure, materials, elements, components, shape, size and arrangement of parts including combinations within the principles of the disclosure, to the full extent indicated by the broad, general meaning of the terms in which the appended claims are expressed. To the extent that these various modifications do not depart from the spirit and scope of the appended claims, they are intended to be encompassed therein.

1. A tablet comprising omeprazole and valacyclovir.

2. The tablet of claim 1, further comprising an enteric coating, wherein the enteric coating is disposed on a surface of the tablet.

3. The tablet of claim 2, wherein the enteric coating comprises a copolymer of methacrylic acid and ethyl acrylate.

4. The tablet of claim 2, wherein the enteric coating dissolves in response to a change in pH, wherein the enteric coating is resistant to dissolution at a pH below about 4 to about 5.5, and wherein the enteric coating dissolves at a pH between about 5 and about 7.5.

5. The tablet of claim 1, wherein the omeprazole is present in an amount of about 10 mg to about 80 mg.

6. The tablet of claim 1, wherein the valacyclovir is present in an amount of about 250 mg to about 3000 mg.

7. The tablet of claim 1, wherein the omeprazole is present in an amount of about 40 mg and the valacyclovir is present in an amount of about 1000 mg.

8. A method for treating a herpes viral infection in a human, the method comprising administering to the human a therapeutically effective amount of a tablet, the tablet comprising omeprazole and valacyclovir.

9. The method of claim 8, wherein the herpes viral infection is selected from the group consisting of herpes simplex virus type 1 infection, herpes simplex virus type 2 infection, and varicella-zoster virus infection.

10. The method of claim 8, wherein the tablet is administered twice daily.

11. The method of claim 10, wherein the tablet is administered for a period of about 3 to about 14 days, more particularly about 5 to about 7 days, and even more particularly about 5 to about 6 days.

12. The method of claim 8, wherein the tablet further comprises an enteric coating, the enteric coating being resistant to dissolution in gastric juices.

13. The method of claim 8, wherein the omeprazole is present in an amount of about 40 mg and the valacyclovir is present in an amount of about 1000 mg.

14. The method of claim 8, wherein the tablet comprises an enteric coating, and wherein the omeprazole and the valacyclovir are absorbed systemically through intestinal mucosa in response to degradation of the enteric coating.

15. A tablet comprising a proton-pump inhibitor and a nucleoside analogue, wherein the proton-pump inhibitor and the nucleoside analogue are disposed within a tablet core at least partially surrounded by an enteric coating.

16. The tablet of claim 15, wherein the proton-pump inhibitor is at least one of omeprazole, esomeprazole, pantoprazole, lansoprazole, rabeprazole, and dexlansoprazole.

17. The tablet of claim 15, wherein the nucleoside analogue is at least one of valacyclovir, acyclovir, famciclovir, and penciclovir.

18. The tablet of claim 15, wherein the enteric coating comprises a copolymer of methacrylic acid and ethyl acrylate.

19. The tablet of claim 15, wherein the proton-pump inhibitor is present in an amount of about 10 mg to about 80 mg.

20. The tablet of claim 15, wherein the nucleoside analogue is present in an amount of about 250 mg to about 3000 mg.
